(19) United States
(12) Patent Application Publication
(10) Pub. No.: US 2022/0110901 A1
(43) Pub. Date: Apr. 14, 2022

(54) FATTY ACID AMIDES AND USES THEREOF IN THE TREATMENT OF NAUSEA

(71) Applicants: Linda PARKER, Campbell River (CA); Erin Michelle Rock, Guelph (CA); Raphael MECHOULAM, Jerusalem (IL)

(72) Inventors: Linda PARKER, Campbell River (CA); Erin Michelle Rock, Guelph (CA); Raphael MECHOULAM, Jerusalem (IL)

(21) Appl. No.: 17/559,074

(22) Filed: Dec. 22, 2021

Related U.S. Application Data
(63) Continuation-in-part of application No. 16/613,213, filed on Nov. 13, 2019.
(60) Provisional application No. 62/509,252, filed on May 22, 2017.
(60) Provisional application No. 63/211,153, filed on Jun. 16, 2021.

(51) Int. Cl. A61K 31/198; A61P 1/08

(52) U.S. Cl. CPC A61K 31/198; A61P 1/08

(57) ABSTRACT

The present invention is directed to a fatty acid amide of an amino acid, including a stereoisomer and a salt thereof for use in the treatment of a patient suffering from nausea, including any condition and symptom associated therewith.

### BACKGROUND OF THE INVENTION

Oleoyl glycine has been identified as an endogenous fatty acid amide released in the insular cortex of mice exposed to traumatic brain injury (Donvito et al. 2019). The insular cortex is a region implicated in nicotine addiction in humans (Naqvi et al. 2007) and rats (Abdolahi et al. 2010). When systemically administered, oleoyl glycine altered withdrawal responses in nicotine dependent mice as well as the rewarding effects of nicotine, as assessed in the conditioned place preference (CPP) paradigm (Donvito et al. 2019). The insular cortex is also essential for the sensation of nausea in humans (Penfield and Faulk 1955; Napadow et al. 2013; Sclocco et al. 2016) and rats as well as morphine withdrawal (MWD) in rats (Li et al. 2009; Li et al. 2013; Wills et al. 2016). Petrie et al. (2019) recently reported that systemic oleoyl glycine prevented acute naloxone-precipitated MWD-induced conditioned place aversion (CPA). Rock et al. (2020) extended this finding by demonstrating that oleoyl glycine also interfered with somatic effects of naloxone-precipitated MWD, including the reaction of conditioned gaping which is a measure of nausea in rats (Grill & Norgren 1978; Parker 2014; McDonald et al. 1997). Oleoyl glycine is a fatty acid amide closely related to anandamide. Anandamide is hydrolyzed rapidly by amidases in the body. In order to stabilize anandamide, Abadji et al. (1994) synthesized methanandamide, in which a methyl group was attached to the carbon atom next to the nitrogen atom of anandamide. Methanandamide possesses a remarkable stability to fatty acid amide hydrolase (FAAH) and has a higher affinity for the cannabinoid 1 (CB1) receptor than anandamide. In order to stabilize oleoyl glycine, the Jerusalem group synthesized the same type of derivative, namely monomethylated oleoyl glycine (oleoyl alanine; HU595) as well as dimethylated oleoyl glycine (HU596), in which one or two methyl groups, respectively, were attached to the carbon atom next to the nitrogen atom. Like oleoyl glycine, oleoyl alanine is found, together with other N-acyl alanines and N-acyl amino acids, in both invertebrate and mammalian tissues, including the mouse brain (Tortoriello et al. 2013; Leishman et al. 2016). We have previously demonstrated that oleoyl glycine prevents the somatic withdrawal responses elicited by acute naloxone-precipitated MWD which include abdominal contractions, lying flattened on belly, diarrhea, mouthing movements, and the nausea-like conditioned gaping reactions in the TR test by a peroxisome proliferator-activated receptor alpha (PPARα) and a cannabinoid 1 (CB1) receptor mechanism of action (Rock et al. 2020). This finding is consistent with the in vitro action of oleoyl glycine as a PPARα agonist and a fatty acid amide hydrolase (FAAH) inhibitor (Donvito et al. 2019). Furthermore, oleoyl glycine (1 and 5 mg/kg, i.p.) prevented the aversive effects of naloxone precipitated MWD in a CPA test (Petrie et al. 2019). Here, we compared the effectiveness of HU595 (monomethylated oleoyl glycine; oleoyl alanine) and HU596 (dimethylated oleoyl glycine) with that of oleoyl glycine in interfering with somatic and affective effects of acute naloxone-precipitated MWD in rats. In experiment 1, we evaluated the potential of oleoyl alanine (HU595; 5 mg/kg, i.p.) to interfere with somatic withdrawal symptoms at the same dose that oleoyl glycine was effective (Rock et al. 2020). Experiment 2 compared the duration of action (10 and 60 min) of oleoyl alanine (5 mg/kg, i.p.) with that of oleoyl glycine (5 mg/kg, i.p.) in preventing naloxone-induced MWD CPA; it also assessed the potential of a lower dose of oleoyl alanine (1 mg/kg i.p.) and a longer duration of action (120 min) to suppress CPA. Experiment 3 ensured that oleoyl alanine (HU595) did not produce CPA or CPP on its own. Experiment 4 evaluated the potential of dimethylated oleoyl glycine (HU596) to interfere with naloxone-precipitated MWD CPA. Having determined that HU595 (oleoyl alanine), but not HU596, interfered with MWD CPA, experiment 5 evaluated the mechanism of action (CB1 receptor and PPARα) of the effect. Experiment 6 evaluated the potential of oleoyl alanine to interfere with decreased saccharin preference following exposure to acute naloxone-precipitated MWD, a commonly used measure of anhedonia in rodents (Scheggi et al. 2018).

Finally, experiment 7 employed the in vitro techniques of competitive activity-based protein profiling (ABPP) to determine if HU595 and HU596 bind with FAAH and luciferase assay to determine if HU595 and HU596 bind with PPAR.

### SUMMARY OF THE INVENTION

The present invention thus provides a fatty acid amide of an amino acid, including a stereoisomer and a salt thereof for use in the treatment of a patient suffering from nausea including any condition and symptom associated therewith.

The term “nausea” should be understood to include any type of diffuse sensation of unease and discomfort, often perceived as an urge to vomit. While not painful, it can be a debilitating symptom if prolonged and has been described as placing discomfort on the chest, upper abdomen, or back of the throat. It should be understood that nausea is a non-specific symptom, having many possible causes. Typically, non-limiting common causes of nausea are gastroenteritis and other gastrointestinal disorders, food poisoning, motion sickness, dizziness, migraine, fainting, low blood sugar and lack of sleep. Additionally, nausea is a side effect of many medications including chemotherapy, or morning sickness in early pregnancy. Nausea may also be caused by disgust and depression.

The compound of the present invention is an antiemetic agent that is effective against vomiting and nausea.

In some embodiments, a compound of the present invention is used to treat motion sickness and the side effects of opioid analgesics, general anaesthetics, and chemotherapy directed against cancer. In some other embodiments, a compound of the invention is used for the treatment of severe cases of gastroenteritis, especially if the patient is dehydrated.

The invention further provides a method of treating nausea including any condition and symptom associated therewith in a patient suffering therefrom, said method comprising administering to said patient a fatty acid amide of an amino acid, including a stereoisomer and a salt thereof.

The invention further provides a fatty acid amide of an amino acid, including a stereoisomer and a salt thereof for use in the treatment of a patient suffering from nausea.

As used herein the term “fatty acid amide of an amino acid” is meant to encompass a compound achieved by the conjugation of a fatty acid moiety (having the general formula —C(═O)R₁, wherein R₁ is as defined herein) and an amino acid moiety (having the general formula —NHCR₂R₃C(═O)OH, wherein R₂ and R₃ are as defined herein) through the formation of an amidic bond between the nitrogen atom of the amino acid moiety (—NHCR₂R₃C(═O)OH) and the carbonylic atom (—C(═O)R₁) of the fatty acid moiety. It should be understood that while compounds of the invention are generally referred to as a conjugate of a fatty acid moiety and an amino acid moiety, the conjugate of the invention may be formed from a variety of precursors, employing a single or multi-step synthetic methodologies.

When referring to a “fatty acid moiety” it should be understood to encompass an acyl moiety derivable from a fatty acid, namely being generally of the form R₁C(═O)—, wherein R₁ represents the aliphatic chain (saturated or unsaturated) of the corresponding fatty acid, and wherein the point of attachment of the fatty acid moiety to the amino acid moiety of the fatty acid amide is through the carbonyl carbon atom of the fatty acid moiety.

As used herein the term “fatty acid” is meant to encompass a mono carboxylic acid having an aliphatic chain (“tail”), wherein said aliphatic chain may be either saturated, mono-unsaturated (having one unsaturated bond anywhere on the aliphatic chain) or poly unsaturated (having at least two unsaturated bonds anywhere on the aliphatic chain). An unsaturated bond on the aliphatic chain may be a double (in the cis and/or trans configuration) or a triple bond. The length of the aliphatic chain (being either saturated, monounsaturated or polyunsaturated) of a fatty acid may vary between 13 to 22 carbon atoms. Fatty acids may be derived from a natural source (either an animal or plant source), synthetic source or semi-synthetic source.

Non-limiting examples of saturated fatty acids are lauric acid, myristic acid, palmitic acid and stearic acid. Non-limiting examples of monounsaturated fatty acids are myristoleic acid, palmitoleic acid and oleic acid. Non-limiting examples of polyunsaturated fatty acids are linoleic acid, α-linolenic acid, arachidonic acid, eicosapentaenoic acid, erucic acid and docosahexaenoic acid.

In some embodiments, said fatty acid moiety of a fatty acid amide is selected from a saturated fatty acid moiety (i.e. R₁ is a hydrocarbon that consists only of single saturated bonds), a mono-unsaturated fatty acid moiety (i.e. R₁ is a hydrocarbon that comprises one unsaturated bond—either a double or triple bond) and a poly unsaturated fatty acid moiety (i.e. R₁ is a hydrocarbon that comprises at least two unsaturated bond—each independently either a double or triple bond). In other embodiments of the invention, the fatty acid moiety is an oleoyl fatty acid moiety (CH₃(CH₂)₇CH═CH(CH₂)₇C(═O)—), namely derived from the corresponding oleic acid.

In some further embodiments, said fatty acid moiety is substituted by at least one group selected from —C₁-C₆ alkyl, —OH, —SH and —SR₂, wherein R₁ and R₂ are each independently —C₁-C₆ alkyl. In other embodiments, said fatty acid moiety is substituted by at least one —C₁-C₆ alkyl. In other embodiments, said fatty acid moiety is substituted by at least two —C₁-C₆ alkyl. In yet other embodiments, said at least one C₁-C₆ alkyl is methyl.

In further embodiments, said at least one substitution is on at least one of the α- or β-positions of said fatty acid moiety. As known in the art, the “α-position of said fatty acid moiety” is the carbon atom on the aliphatic chain of the fatty acid moiety which is directly adjacent to the carbonyl carbon atom of the fatty acid moiety; the “β-position of said fatty acid moiety” is the carbon atom on the aliphatic chain of the fatty acid moiety which is the second carbon atom adjacent to the carbonyl carbon atom of the fatty acid moiety.

In some embodiments, a fatty acid amide of the invention is substituted at the α-position of the fatty acid moiety. In other embodiments, a fatty acid amide of the invention is substituted at the β-position of the fatty acid moiety. In further embodiments, a fatty acid amide of the invention is substituted at both the α- and β-positions of the fatty acid moiety.

When referring to an “amino acid moiety” it should be understood to encompass a radical derivable from an amino acid, namely being generally of the formula —NHCR₂R₃COOH, wherein the point of attachment of said amino acid moiety to a fatty acid moiety, as defined herein, is through the amine of the amino acid moiety, as explained above.

The “amino acid” is an amino acid (i.e., alpha-amino acid or beta-amino acid) as known in the art. In some embodiments, the amino acid moiety is derived from an amino acid of the general formula H₂NCR₂R₃COOH, wherein R₂ and R₃ are as defined above. Non-limiting examples of amino acids which correspond to the amino acid moiety of a compound defined herein are alanine, asparagine, aspartate, cysteine, glutamate, glutamine, glycine, dimethylglycine, proline, serine, tyrosine, arginine, histidine, isoleucine, leucine, lysine, methionine, phenylalanine, threonine, tryptophan, and valine. An amino acid as used herein may be derived from of a natural source, synthetic or semi-synthetic source. An amino acid as used herein may also be in the D- or L-configuration. In some embodiments, an amino acid is an L-amino acid.

In some embodiments, said amino acid moiety is selected from serine, glycine, dimethylglycine, alanine, cysteine, tyrosine and phenylalanine. In other embodiments, said amino acid moiety is serine.

In some embodiments, of the invention said fatty acid moiety is optionally substituted by one group selected from —C₁-C₆ alkyl, —OH, —O(C₁-C₁₀ alkyl), —SH and —S(C₁-C₁₀ alkyl); and the amino acid moiety is optionally substituted by one group selected from —C₁-C₆ alkyl, —OH and —O(C₁-C₁₀ alkyl), phenyl and phenol.

In further embodiments, said amino acid moiety is unsubstituted.

In still further embodiments, said amino acid moiety is substituted by at least one group selected from —C₁-C₆ alkyl, —OH, and —O(C₁-C₁₀ alkyl), wherein R₃ is —C₁-C₆ alkyl. In other embodiments, said amino acid is substituted by at least one —C₁-C₆ alkyl. In other embodiments, said amino acid is substituted by at least two —C₁-C₆ alkyl. In further embodiments, said —C₁-C₆ alkyl is methyl. In yet additional embodiments, said substitution is on the α-position of said amino acid moiety.

The “α-position of said amino acid moiety” is the carbon atom on the amino acid moiety which is directly adjacent to the carbonyl carbon atom of the amino acid moiety.

In some further embodiments, said amino acid moiety is selected from a moiety of serine, cysteine, glycine, dimethylglycine, alanine, tyrosine and phenylalanine. In some embodiments, said amino acid moiety is substituted by at least one group selected from straight or branched —C₁-C₆ alkyl, straight or branched —C₂-C₆ alkenyl, straight or branched —C₂-C₆ alkynyl, —OH, and —O(C₁-C₁₀ alkyl).

In some other embodiments, said amino acid is substituted by at least one —C₁-C₆ alkyl. In other embodiments, said amino acid is substituted by at least two —C₁-C₆ alkyl. In yet further embodiments, said —C₁-C₆ alkyl is methyl. In some embodiments, said substitution is on the α-position of said amino acid moiety.

In some embodiments, said fatty acid moiety is substituted by at least one group selected from —C₁-C₆ alkyl, —OH, —O(C₁-C₁₀ alkyl), —SH and —S(C₁-C₁₀ alkyl). In further embodiments, said fatty acid moiety is substituted by at least one —C₁-C₆ alkyl. In some embodiments, at least one C₁-C₆ alkyl is methyl. In further embodiments, said at least one substitution is on at least one of α- or β-positions of said fatty acid moiety.

In some embodiments, a fatty acid amide of the invention is a compound of general formula (I), including a stereoisomer and a salt thereof:

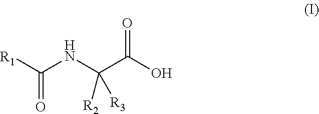

Wherein R₁ is selected from straight or branched —C₁₃-C₂₂ alkyl, straight or branched —C₁₃-C₂₂ alkenyl and straight or branched —C₁₃-C₂₂ alkynyl; optionally substituted by at least one group selected from —C₁-C₆ alkyl, —OH, —O(C₁-C₁₀ alkyl), —SH and —S(C₁-C₁₀ alkyl); R₂ and R₃ are independently selected from H, straight or branched —C₁-C₆ alkyl, straight or branched —C₂-C₆ alkenyl, straight or branched —C₂-C₆ alkynyl; each optionally substituted by at least one —OH, —SH, —O(C₁-C₆ alkyl), phenyl and phenol; provided that at least one of R₂ and R₃ is different than H.

In some embodiments, R₂ is —C₁-C₆ alkyl. In other embodiments, R₃ is —C₁-C₆ alkyl. In further embodiments, R₂ and R₃ are each independently —C₁-C₆ alkyl. In yet other embodiments, said —C₁-C₆ alkyl is methyl. In some embodiments, R₁ is a —C₁₃-C₂₂ alkenyl. In some embodiments, said —C₁₃-C₂₂ alkenyl comprises between 1 to 6 double bonds.

The term “stereoisomer” as used herein is meant to encompass an isomer that possess identical constitution as a corresponding stereoisomer, but which differs in the arrangement of its atoms in space from the corresponding stereoisomer. For example, stereoisomers may be enantiomers, diastereomers and/or cis-trans (E/Z) isomers. It should be understood that a composition comprising a fatty acid amide of the invention may comprise single enantiomers, single diastereomers as well as mixtures thereof at any ratio (for example racemic mixtures, non racemic mixtures, mixtures of at least two diastereomers and so forth). Furthermore, the invention encompasses any stereoisomer of a fatty acid amide of the invention achieved through in vivo or in vitro metabolism, or by any type of synthetic rout.

The term “salt” as used herein is meant to encompass any salt achieved by acid or base addition. In some embodiments, the salt is an acid addition salt obtained by protonation of a fatty acid amide of the invention (for example at the amidic moiety). In other embodiments, the salt is a base addition salt obtained by deprotonation of a proton from the fatty acid amide of the invention (for example from the acidic moiety, i.e. —COOH of the fatty acid amide). Counter ion forming a salt of a fatty acid amide of the invention can, in a non-limiting fashion, include inorganic or organic cations, which in some embodiments are pharmaceutically acceptable, such as alkaline metal cations e.g. potassium or sodium cation, alkaline earth metal cations such as magnesium or calcium, or ammonium cation including e.g. the cations derived from an organic nitrogen-containing base, such as trialkylamine-derived cations for example triethylammonium ion.

The term “alkyl” is meant to encompass a monovalent linear (unbranched), branched or cyclic saturated hydrocarbon radical. When referring to “C₁-C₆ alkyl” it should be understood to encompass any linear or branched alkyl having 1, 2, 3, 4, 5, or 6 carbon atoms. Non-limiting examples of C₁-C₆ alkyl include methyl, ethyl, n-propyl, iso-propyl, n-butyl, 2-butyl, 3-butyl, n-isobutyl, 2-isobutyl, n-pentyl, 2-pentyl, 3-pentyl, 2-methylbutyl, 2-dimethylpropyl, n-hexyl, 2-hexyl, 3-hexyl, 2-methylpentyl, 3-methylpentyl, 2,3-dimethylbutyl, 2,2-dimethylbutyl, 2-methyl-2-ethyl-propyl, cyclobutyl, 1-methyl-cyclobutyl, 2-methyl-cyclobutyl, 1,1-dimethyl-cyclobutyl, 1,2-dimethyl-cyclobutyl, dimethyl-cyclobutyl, methyl-1-cyclobutyl, 1-cyclobutyl-ethyl, 2-cyclobutyl-ethyl, cyclopentyl, 1-methyl-cyclopentyl, 2-methyl-cyclopentyl. Similarly, when referring to “—C₁₁-C₂₀ alkyl” it should be understood to encompass any linear or branched alkyl radical having 11, 12, 13, 14, 15, 16, 17, 18, 19, 20 carbon atoms. Similarly, when referring to “—C₁₃-C₂₂ alkyl” it should be understood to encompass any linear or branched alkyl radical having 13, 14, 15, 16, 17, 18, 19, 20, 21, 22 carbon atoms.

The term “alkenyl” is meant to encompass a linear (unbranched) or branched hydrocarbon chain having at least one double bond. A double bond may be between any two carbon atoms of the alkenyl chain and may be in the cis or trans (or the E or Z) configuration. A double bond of an alkenyl may be unconjugated or conjugated to another unsaturated group. When referring to “—C₁₃-C₂₂ alkenyl” it should be understood to encompass any linear or branched alkenyl radical having 13, 14, 15, 16, 17, 18, 19, 20, 21, 22 carbon atoms. Similarly, when referring to “—C₁₁-C₂₀ alkenyl” it should be understood to encompass any linear or branched alkenyl radical having 11, 12, 13, 14, 15, 16, 17, 18, 19, 20 carbon atoms.

The term “alkynyl” is meant to encompass a linear (unbranched) or branched hydrocarbon chain having at least one triple bond. The triple bond may be between any two carbon atoms of the alkynyl chain. The triple bond of an alkynyl may be unconjugated or conjugated to another unsaturated group. When referring to “—C₁₃-C₂₂ alkynyl” it should be understood to encompass any linear or branched alkynyl radical having 13, 14, 15, 16, 17, 18, 19, 20, 21, 22 carbon atoms. Similarly, when referring to “—C₁₁-C₂₀ alkynyl” it should be understood to encompass any linear or branched alkynyl radical having 11, 12, 13, 14, 15, 16, 17, 18, 19, 20 carbon atoms.

The term “phenyl” should be understood to mean the aromatic cyclic group having the formula C₆H₅. The term “phenol” should be understood to mean the aromatic group having the formula C₆H₄OH, wherein said —OH group may be substituted at any point on the cyclic ring.

Certain of the above defined terms may occur more than once in the structural formulae, and upon such occurrence each term shall be defined independently of the other.

The term “optionally substituted” as used herein means that the groups in question are either unsubstituted or substituted with one or more of the substituents specified. When the groups in question are substituted with more than one substituent, the substituents may be the same or different.

In another aspect, the invention encompasses a pharmaceutical composition comprising a fatty acid amide as disclosed herein including any stereoisomer and salt thereof. The invention further provides a pharmaceutical composition comprising at least one fatty acid amide as disclosed herein including any stereoisomer and salt thereof, in combination with at least one other therapeutic agent. The invention further provides a use of a fatty acid amide disclosed herein for the preparation of a pharmaceutical composition.

The present invention also relates to a pharmaceutical composition comprising a fatty acid amide disclosed herein in combination (e.g., admixture) with a pharmaceutically acceptable auxiliary, and optionally at least one additional therapeutic agent. The auxiliary must be “acceptable” in the sense of being compatible with the other ingredients of the composition and not deleterious to the recipients thereof.

Pharmaceutical compositions include those suitable for oral, rectal, nasal, topical (including transdermal, buccal and sublingual), vaginal or parenteral (including subcutaneous, intramuscular, intravenous and intradermal) administration or administration via an implant.

In some embodiments a pharmaceutical composition disclosed herein is a transdermal composition. In some other embodiments said fatty acid amide disclosed herein is administered to a patient using a transdermal formulation. In some embodiments, said transdermal formulation/composition employs the use of a dermal patch.

In some embodiments a pharmaceutical composition disclosed herein is a nasal composition. In some other embodiments said fatty acid amide disclosed herein is administered to a patient using a nasal formulation. In some embodiments, said nasal formulation/composition employs the use of a delivery device (for example a nebulizer).

The compositions may be prepared by any method well known in the art of pharmacy. Such methods include the step of bringing in association fatty acid amides of the invention or combinations thereof with any auxiliary agent. The auxiliary agent(s), as the accessory ingredient(s), is typically selected from those conventional in the art, such as carriers, fillers, binders, diluents, disintegrants, lubricants, colorants, flavouring agents, anti-oxidants, and wetting agents.

Pharmaceutical compositions suitable for oral administration may be presented as discrete dosage units such as pills, tablets, dragées or capsules, or as a powder or granules, or as a solution or suspension. The active ingredient may also be presented as a bolus or paste. The compositions may further be processed into a suppository or enema for rectal administration.

The invention further includes a pharmaceutical composition, as hereinbefore described, in combination with packaging material, including instructions for the use of the composition for a use as hereinbefore described.

For parenteral administration, suitable compositions include aqueous and non-aqueous sterile injection. The compositions may be presented in unit-dose or multi-dose containers, for example sealed vials and ampoules, and may be stored in a freeze-dried (lyophilised) condition requiring only the addition of sterile liquid carrier, for example water, prior to use. For transdermal administration, e.g. gels, patches or sprays can be contemplated. Compositions or formulations suitable for pulmonary administration e.g. by nasal inhalation include fine dusts or mists which may be generated by means of metered dose pressurized aerosols, nebulisers or insufflators.

The exact dose and regimen of administration of the composition will necessarily be dependent upon the effect to be achieved and may vary with the particular formula, the route of administration, and the age and condition of the individual subject to whom the composition is to be administered.

The invention further provides a kit comprising at least one compound of the invention or a pharmaceutical composition comprising thereof, as hereinbefore described, and instructions for use thereof.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 shows the structure of oleoyl glycine, monomethyl oleoyl glycine (oleoyl alanine, HU595), and dimethyl oleoyl glycine (HU596).

FIG. 2 shows the mean (±SEM) seconds spent on the saline-paired floor and the MWD-paired floor for each pretreatment group (VEH, oleoyl glycine, and HU595 (oleoyl alanine) at each pretreatment time in experiment 2. ***p<0.001, *p<0.05 difference between floor preference.

FIG. 3 shows the mean (±SEM) seconds spent on the saline-paired floor and the MWD-paired floor in experiment 2. Dimethylated oleoyl glycine (HU596) did not modify the strength of the CPA. **p<0.01.

FIG. 4 shows the mean (±SEM) seconds spent on the saline-paired floor and the MWD-paired floor in experiment 5 in which rats were pretreated with MK886 (1 mg/kg, i.p.) or AM251 (1 mg/kg, i.p.) prior to treatment with HU595 (5 mg/kg, i.p.). Asterisks indicate significance, with **p<0.01; **p<0.001.

FIG. 5 shows the mean (±SEM) cumulative saccharin preference ratios by group across the two-bottle saccharin preference testing period for both VEH and HU595 pretreated animals. Two asterisks indicate a significant difference between groups (p<0.01).

FIGS. 6 a and b (top section) shows the in vitro competitive affinity-based proteome profiling (ABPP) of oleoyl glycine, oleoyl alanine (HU595), and HU596 using the serine hydrolase-directed probe FP-Rh in the membrane fraction of the rat brain proteome. FIG. 6a The selectivity profiles of oleoyl glycine, HU596, and HU595 (10-30 μM), as judged by competitive ABPP analysis in the rat brain membrane proteome. FIG. 6b Quantification of % of FAAH inhibition determined by measuring fluorescent intensity of gel band using the ImageJ 1.43u software. Data represent the mean±SD of n=2 separate experiments. c and d (bottom section) Luciferase assays in PPARα-transfected COS-7 cells. Bar graphs show the concentration dependent effect of FIG. 6c oleoyl alanine (HU595) and FIG. 6d HU596 in comparison with fenofibrate, in COS-7 cells transiently transfected with human PPARα-Gal 4 plasmid. Cells were co-transfected with equimolar amounts of plasmid encoding for TK-MH100×4-Luc containing the UAS enhancer elements and Renilla luciferase as transfection efficiency control and normalize the signal intensity of luciferase. HU595 and HU596 were dissolved in DMSO (vehicle, 0.003%). Data are expressed as the mean±SEM of four independent determinations. *p<0.05 vs control group, determined by Student's t test.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

Materials and Methods

Animals

The 209 subjects were male Sprague Dawley rats (200 to 250 g on arrival in the laboratory) purchased from Charles River Labs, St. Constant, Quebec. Animals were pair-housed in an opaque Plexiglas cage while receiving food and water ad libitum, except in experiment 6 in which animals were single housed with food and water ad libitum. They were exposed to a 12/12 h reverse light/dark cycle where the lights turn on at 7 p.m. All experiments were conducted during the rats' dark cycle. The colony room housing all of the rats was kept at 21° C. All animal procedures were approved by the Animal Care Committee of the University of Guelph and adhere to the guidelines of the Canadian Council of Animal Care.

Drugs

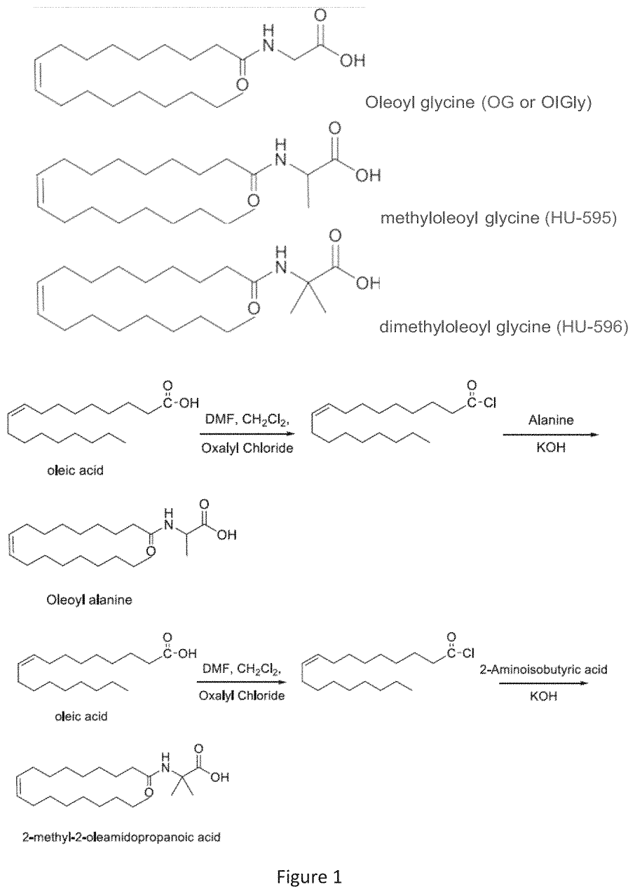

Morphine and naloxone (Ontario Veterinary College (OVC) pharmacy) were prepared with saline at a concentration of 20 and 1 mg/ml, respectively, before injecting subcutaneously (s.c.) at a volume of 1 ml/kg. Oleoyl glycine (prepared by the Jerusalem group as previously described (Donvito et al. 2019)), HU595 (oleoyl alanine; monomethylated oleoyl glycine), and HU596 (dimethylated oleoyl glycine) were dissolved in a vehicle mixture of ethanol, Tween 80, and physiological saline in a 1:1:18 ratio. When oleoyl glycine, oleoyl alanine (HU595), and HU596 were first dissolved in ethanol, Tween 80 was then added to the solution, and the ethanol was evaporated off with a nitrogen stream; after which, the saline was added. The final vehicle (VEH) consisted of 1:9 (Tween/saline). Oleoyl glycine was prepared as a 5 mg/ml solution; oleoyl alanine (HU595) and HU596 (see FIG. 1 for molecular structure of each) were prepared as 1 mg/ml and 5 mg/ml solutions; all drugs were administered i.p. at a volume of 1 ml/kg. Lithium chloride (LiCl; Sigma Aldrich) was prepared in a 0.15 M solution with sterile water and was administered i.p. at a volume of 20 ml/kg (127.2 mg/kg dose). Synthesis of monomethyl oleoyl glycine (oleoyl alanine; HU595) Oxalyl chloride (2.0 M solution in methylene chloride, 3.5 ml, 7 mmol) was added dropwise under nitrogen atmosphere to a solution of oleic acid (1 g, 3.54 mmol) and N,N-dimethylformamide (266 ″1, 3.64 mmol) in dry methylene chloride (10 ml). The reaction mixture was stirred for 1 h and then the solvent was evaporated under a nitrogen flow. The crude material in methylene chloride (10 ml) was added to a solution of alanine (945 mg, 10.62 mmol) and 2 N potassium hydroxide in an ice bath. Then, the reaction mixture was stirred for 1 h, water (10 ml) was added, and the mixture was acidified to pH 3 with 1 N HCl. The product was extracted with ether (3×50 ml) and dried (MgSO4), and solvent was evaporated under reduced pressure. The crude material was chromatographed on silica gel (eluting with chloroform:methanol) to yield white powder. Melting point 42° C.; NMR (CDCl3, ppm) 6.11-6.01 (d, 1H), 5.35-5.32 (m, 2H), 4.62-4.51 (m, 1H), 2.43 (bs, 1H), 2.25-2.20 (t, 2H), 2.01-1.97 (m, 4H), 1.66-1.60 (m, 2H), 1.46-1.44 (d, 3H), 1.30-1.26 (m, 20H), 0.89-0.85 (t, 3H (LC-MS (—H) 352 m/z. Synthesis of dimethyl oleoyl glycine (HU596) Oxalyl chloride (2.0 M solution in methylene chloride, 3.5 ml, 7 mmol) was added dropwise under nitrogen atmosphere to a solution of oleic acid (1 g, 3.54 mmol) and N,N-dimethylformamide (266 ″1, 3.64 mmol) in dry methylene chloride (10 ml). The reaction mixture was stirred for 1 h and then the solvent was evaporated under a nitrogen flow. The crude material in methylene chloride (10 ml) was added to a solution of 2-aminoisobutyric acid (1.1 g, 10.62 mmol) and 2 N potassium hydroxide in an ice bath. Then, the reaction mixture was stirred for 1 h, water (10 ml) was added, and the mixture was acidified to pH 3 with 1 N HCl. The product was extracted with ether (3×50 ml) and dried (MgSO4), and solvent was evaporated under reduced pressure. The crude material was chromatographed on silica gel (eluting with chloroform:methanol) to yield a yellowish powder. NMR (CDCl3, ppm) 5.42 (m, 2H), 2.18-2.13 (m, 6H), 1.56 (s, 6H), 1.53 (m, 2H), 1.31-1.29 (m, 20H), 0.88 (t, 3H (LCMS (—H) 366 m/z.

Apparatus

In experiment 1, for investigation of the acute naloxone precipitated MWD somatic reactions, the observation chambers consisted of 4 black Plexiglas boxes (22.5×26×20 cm) with an opaque lid, sitting on top of a clear glass-topped table. A closed-circuit Panasonic WV-CP484 video camera was placed below each of the chambers to video record somatic withdrawal behaviors that were FireWired to a computer for later scoring using “The Observer” event recording software (Noldus Information Technology Inc., Leesburg, Va.). For investigation of conditioned gaping reactions in experiment 1, the rats were placed in taste reactivity (TR; Grill and Norgren 1978) chambers with their cannula attached to an infusion pump (Model KDS100, KD Scientific, Holliston, Mass., USA) for fluid delivery. The TR chamber was in a dark room next to a 25-W light source. The TR chambers used were made of clear Plexiglas (22.5×26×20 cm) and placed on a table with a clear glass top. A mirror beneath the chamber at a 45° angle facilitated viewing of the ventral surface of the rat to observe orofacial responses. A Sony video camera (Handycam, Henry's Camera; Waterloo, ON) with a FireWire connection to a computer was focused on the mirror and used to record the rats from the mirror beneath the chamber. The videos were later scored using The Observer. For experiments 2-5, a place conditioning apparatus with removable floors was used as described by Wills et al. (2016). The conditioning apparatus was a rectangular box (60×25×25 cm) made of black Plexiglas and a wire mesh lid. During conditioning, removable metal floors characterized by either a hole surface (1 cm in diameter spaced 1 cm apart from each other) or a grid surface (½ cm horizontal bars spaced 1 cm apart) were placed upon a black rubber mat on top of the black Plexiglas surface. The different floors serve as contextual cues that differentiate the treatment floor from the VEH floor. During the test and pretest trials, black metal floors split into two equal halves (half hole and half grid surface) were placed into the conditioning boxes. The tactile stimulus properties of the two floor halves were identical to their matching floor counterparts used in conditioning. EthoVision software (Noldus, Inc., Netherlands) was used to automatically capture the movement of the rat among the floors, which was collected by a video camera attached to the ceiling. For experiment 6, assessment of saccharin preference was conducted in the home cage in the colony room. Bottles with sipper tubes were inserted into the home cage with the saccharin-containing bottle placed on the right side of the cage across all rats.

Surgery

In experiment 1, all 16 rats, under isoflurane anesthesia, were surgically implanted with an intraoral cannula according to the procedure described by Limebeer et al. (2010).

Procedures

### Experiment 1: Potential of Oleoyl Alanine (HU595) to Prevent Acute Naloxone-Precipitated MWD Somatic Effects in Rats

Following 3 days of recovery from intraoral surgery, the rats received an adaptation trial in which they were placed in the TR chamber with their cannula attached to an infusion pump (Model KDS100, KD Scientific, Holliston, Mass., USA) for fluid delivery. Water was infused into their intraoral cannula for 2 min at the rate of 1 ml/min. The conditioning procedure began 2 h following the adaptation trial. Each rat received two conditioning trial cycles separated by 48 h during which saccharin was paired with acute naloxone-precipitated MWD and somatic reactions were monitored. On the first trial of each cycle, all rats were injected with morphine (20 mg/kg, s.c.) and were placed in an empty Plexiglas cage in the colony room. They were monitored for signs of respiratory distress and returned to their home cage once fully ambulatory. On the second day of each cycle, 24 h after the morphine injection, the rats were injected i.p. (1 ml/kg) with VEH (n=8) or HU595 (5 mg/kg; n=8) and 20 min later (see Rock et al. 2020) were placed in the TR chamber and intraorally infused with 0.1% saccharin solution for 2 min at the rate of 1 ml/min. Immediately after the saccharin infusion, the rats were injected with naloxone (1 mg/kg, s.c.) and were then placed in the somatic withdrawal observation chamber for 30 min. On the second naloxone-precipitated MWD trial, the rats were videotaped for somatic reactions (mouthing movements, abdominal contractions, lying flattened on belly) and instances of diarrhea were recorded (yes or no). Body weight was measured 2 h post naloxone to determine percentage body weight loss. Seventy-two hours following the final MWD trial, the rats were given a 2-min drug-free TR test during which they were intraorally infused with saccharin at the rate of 1 ml/min and their orofacial reactions were recorded.

### Experiment 2: Potential of Oleoyl Glycine and Oleoyl Alanine (HU595) to Prevent Acute Naloxone Precipitated MWD-Induced CPA at 10 Min and 60 Min Prior to Naloxone

The rats (n=96) were placed in the CPA testing apparatus for a 10-min drug-free pretest trial to measure baseline floor preferences. EthoVision software tracked the movement of rats from a video camera in the ceiling throughout the trial to determine how much time was spent on each floor. There were no significant differences in time spent on the hole or the grid floor in any experiment. Floors and conditioning boxes were washed between each trial. One day following the pretest, the rats received two 3-day conditioning trial cycles in order to produce naloxone precipitated MWD-induced CPA (as described by Petrie et al. 2019). On day 1 of each cycle, the floor opposite to the assigned drug floor was paired with s.c. saline injection. The rats were injected i.p. with the VEH and 10 min later were injected i.p. with saline. Ten minutes after a saline injection, the rats were placed into the conditioning box with the assigned saline-paired floor for 20 min. On day 2 of each cycle, the rats received a high dose of morphine (20 mg/kg, s.c.), 24 h after the saline conditioning trial the previous day. After the injection, rats were placed in an empty Plexiglas cage and monitored for signs of respiratory distress and stimulated when necessary until they recovered and were returned to the home cage. On day 3 of the cycle, 24 h post morphine injections, the rats were injected i.p. with VEH, 5 mg/kg oleoyl glycine, or 5 mg/kg HU595 (oleoyl alanine), 10 or 60 min prior to receiving an injection of naloxone (1 mg/kg, s.c.). Ten minutes later, they were placed into the conditioning box with the assigned naloxone-paired floor for 20 min. Four days later, all rats underwent a second 3-day conditioning cycle. Five days following the last naloxone trial, a 10-min drug-free test trial was given. The test trial consisted of the same procedures as the pretest trial, but the rats were given s.c. saline injection 10 min prior to the test. During the test trial, EthoVision tracked the amount of time the rats spent on each floor surface. The groups (n=12/group) were VEH-10 min, VEH-60 min, oleoyl glycine-10 min, oleoyl glycine-60 min, HU595-10 min, and HU595-60 min. To further investigate the duration of effectiveness of oleoyl alanine, an additional group was administered 5 mg/kg i.p., HU595 (n=12), 120 min prior to receiving naloxone over each of the two conditioning trials. Finally, because oleoyl glycine interfered with MWD-induced CPA at a lower dose of 1 mg/kg, i.p. (Petrie et al. 2019), an additional group (n=12) was administered a dose of 1 mg/kg, i.p., of HU595 10 min prior to naloxone on both conditioning trials.

### Experiment 3: Potential of Oleoyl Alanine (HU595) to Produce CPA or CPP on its Own

In order to determine if oleoyl alanine produced CPP or CPA on its own, rats (n=12) received two conditioning trial cycles (as in experiments 2 and 3) with HU595 and VEH beginning the day following a 10-min pretest. On each conditioning trial cycle, they received 1 ml/kg i.p. injections of HU595 (5 mg/kg) or VEH (24 h apart; with VEH and HU595 trials in counterbalanced order) 20 min (Petrie et al. 2019) prior to placement in the conditioning box lined with the grid or hole floor (counterbalanced) for 20 min, while their locomotion was tracked by EthoVision. Three days after the final conditioning day, the rats received a 10-min drug-free as described previously.

### Experiment 4: Potential of Dimethyloleoyl Glycine (HU596) to Interfere with MWD CPA

The rats (n=32) were treated as in experiment 2, except that they were pretreated with VEH (n=12), 1 mg/kg HU596 (n=8), or 5 mg/kg HU596 (n=12) 10 min prior to naloxone.

### Experiment 5: Mechanism of Action of Oleoyl Alanine (HU595) Interference with Acute Naloxone-Precipitated MWD-Induced CPA

The 33 rats were treated as in experiment 2 (with only the 10 min pretreatment time) except that, on the naloxone trials, they also received an injection of VEH, MK886 (1 mg/kg, i.p.; PPARα antagonist), or AM251 (1 mg/kg, i.p.; CB1 receptor antagonist) 20 min prior to an injection of HU595 (5 mg/kg, i.p.). Ten minutes following pretreatment with HU595, the rats were injected with naloxone (1 mg/kg, s.c.) and 10 min later were placed in the MWD chamber. The groups were VEH-HU595 (n=10), MK886-HU595 (n=11), and AM251-HU595 (n=12). Petrie et al. (2019) previously demonstrated that at the doses employed here neither AM251 nor MK886 modified the strength of CPA produced by acute naloxone-precipitated MWD.

### Experiment 6: Effect of Oleoyl Alanine (HU595) on Saccharin Preference Following Acute Naloxone-Precipitated MWD

In order to familiarize the rats with saccharin, the rats (n=80) were pre-exposed to two bottles (saccharin (0.1%) and reverse osmosis water) in their home cage and consumption of both liquids was measured at 2 h, 4 h, 6 h, 8 h, 12 h, and 24 h. The cumulative amount of saccharin and water consumed at each of the six intervals was converted into a cumulative saccharin preference ratio (PR) (cumulative saccharin consumed/(cumulative saccharin+cumulative water consumed)). Using pretest PR scores, rats were assigned to one of 4 groups (N=20/group): saline-saline, saline-naloxone, morphine-saline, or morphine-naloxone. Seventy-two hours following the pretest, the rats received two cycles of acute naloxone-precipitated MWD. On day 1, rats were injected s.c. with saline or 20 mg/kg morphine and were monitored in empty cages for signs of respiratory distress. Twenty four hours later, the rats in each group were pretreated with an i.p. injection of VEH (n=10) or 5 mg/kg HU595 (n=10) 10 min prior to receiving a s.c. injection of saline or naloxone. Over the next 48 h, this cycle was repeated once more, except 10 min following the final saline or naloxone injection where all rats received two bottles (saccharin (0.1%) and reverse osmosis water) in their home cage and consumption of both liquids was measured at the same intervals used during pretest. As in the pretest, the amounts consumed were converted to saccharin PRs.

### Experiment 7: Effect of Oleoyl Alanine (HU595) and HU596 on the Rat Serine Hydrolase Proteome for FAAH Activity and on Luciferase Assay for PPARα Activity Competitive Activity-Based Protein Profiling (ABPP) Assay of Activity at FAAH

For preparation of proteomes, rat brains were dounced homogenized in an isotonic buffer consisting of 20 mM HEPES, 2 mM DTT, 0.25 M sucrose, and 1 mM MgCl2, pH 7.2. Lysed proteomes were then subjected to a low-speed spin (1400×g, 5 min) to remove debris, and ultracentrifugation (100,000×g, 45 min) to separate membrane and cytosolic fractions. The supernatant was removed and saved as the soluble proteome, while the pellet was washed and resuspended in isotonic resuspension solution (20 mM HEPES, 2 m MDTT) by pipetting and saved as the membrane proteome. Rat brain membranes were diluted to 1 mg/ml prior to use (Chang, et al. 2012). Proteomes (50 ″1) were preincubated with either DMSO or 10-30 μM concentrations of oleoyl glycine (OG), HU595, and HU596 at 37° C. After 20 min, FP-Rh (1.0 μl, 50 μM in DMSO, a kind gift from Ben Cravatt) was added and the mixture was incubated for another 30 min at 37° C. Reactions were quenched with SDS loading buffer (12.5 μl, 5×) and run on SDS-PAGE (sodium dodecyl sulfate polyacrylamide gel electrophoresis; Chang et al., 2013). Following gel imaging, serine hydrolase activity was determined by measuring fluorescent intensity of gel band corresponding to FAAH using ImageJ 1.43u software.

Luciferase Assay of PPARα Activity Cell Culture, Reagents, and Transfections

Fibroblast-like (COS-7) cells were propagated in Dulbecco's modified Eagle medium (DMEM; Cat. no. 11965092, Life Technology, Milan, Italy) supplemented with 10% FBS, and 1% penicillin and streptomycin (Cat. no. 15070063, Life Technology, Milan, Italy) in a humidified atmosphere of 95% air/5% CO2 at 37° C. For cell transfection, cells were seeded onto 24-well plastic plates at 2×103 cells/cm2 density. After plating, the cells were transfected on the next day with (a) pM1-hPPARα-Gal4, (b) TK-MH100×4-Luc containing the UAS enhancer elements, and (c) Renilla luciferase (pRL, cat. E2231; Promega, Milan, Italy). The combination of plasmids was transfected into the cells by use of Lipofectamine LTX (Life Technology, Milan, Italy) following the manufacturer's instruction. After 24 h, the cells were treated with vehicle (DMSO 0.003%) and HU595 and HU596 (up to 50 μM).

Luciferase Assay

After 18 h of treatment, the cells were harvested and processed for the luciferase and Renilla luciferase (Promega) using the Dual-Luciferase Reporter Assay System (Promega, cat. E1910) and detected using the GloMax Luminometer (Promega).

Data Analysis

For experiment 1, somatic opioid withdrawal behaviors demonstrated by VEH and oleoyl alanine groups were each entered into separate independent sample t tests. For experiment 2, separate 2×2 mixed-factors ANOVAs were performed for each pretreatment group (VEH, OlGly, HU595) at each time (10 or 60 min) as a between-subjects factor and floor (grid or hole) as a within subjects factor. Also, the data for each of the two additional groups were entered into a paired t test for each floor to determine if oleoyl alanine prevented the MWD-induced CPA at a dose of 1 mg/kg and at a delay of 120 min. For experiment 3, the amount of time spent on the VEH- and HU595-paired floors was entered into a paired sample t test during the test of place preference. The distance traveled on the VEH and oleoyl alanine (HU595) conditioning trials was entered into a 2×2 repeated measures ANOVA with drug (HU595 or VEH) and conditioning trials (1 and 2) as within-subjects factors. For experiment 4, a 3×2 mixed-factors ANOVA was performed with the between subjects factor of pretreatment (VEH 1 mg/kg or 5 mg/kg HU596) and the within-subjects factor of floor (grid or hole). For experiment 5, time spent on each floor was entered into a 3×2 mixed-factors ANOVA with pretreatment (VEH, 1 mg/kg MK886, 1 mg/kg AM251) as a between-subjects factor and floor (grid or hole) as a within-subjects factor. For experiment 6, the PR scores during the test were entered into a 2 (pretreatment, VEH or HU595)×4 (group SS, SN, MS, MN)×6 (time 2 h, 4 h, 6 h, 8 h, 12 h, and 24 h) mixed-factors ANOVA. In experiment 7, the differences among treatments for the luciferase assay were evaluated by t tests.

Results

### Experiment 1: Potential of Oleoyl Alanine (HU595) to Interfere with Somatic Effects of MWD and Conditioned Gaping in Rats

Oleoyl alanine (HU595) interfered with the naloxone precipitated somatic MWD effects of abdominal contractions, lying on belly, and mouthing movements, but not diarrhea or body weight loss. Also, oleoyl alanine, like oleoyl glycine, interfered with MWD-induced conditioned gaping reactions in rats, reflective of conditioned nausea. Table 1 presents the mean (±SEM) somatic MWD reactions and conditioned gaping in the TR test for the groups pretreated with VEH or HU595 prior to naloxone. Independent t tests revealed that the group pretreated with oleoyl alanine displayed significantly fewer MWD-elicited mouthing movements, abdominal contractions, and bouts of lying on belly following the second naloxone injection and displayed significantly fewer conditioned gaping reactions during the drug-free TR test than did the group pretreated with VEH (ps<0.01). However, the groups did not significantly differ in the instances of diarrhea or in percentage body weight loss.

TABLE 1
Acute naloxone-precipitated MWD behaviors in experiment 1

| MWD behavior | VEH pretreatment | | HU595 pretreatment | Statistical significance |
|---|---|---|---|---|
| Mouthing movements (f) | 57.2 | (±6.6) | 30.8 (±7.4) | t (14) = 2.6; p = 0.025 |
| Abdominal contractions (f) | 13.1 | (±2.4) | 5.0 (±0.8) | t (14) = 3.2; p = 0.006 |
| Lying on belly (s) | 722.3 | (±120.4) | 102.0 (±57.9) | t (14) = 4.6; p < 0.001 |
| Diarrhea (yes/no) | 0.5 | (±0.2) | 0.8 (±0.2) | t (14) = 1.0; ns |
| % Body weight loss | 2.7 | (±0.5) | 2.4 (±0.4) | t (14) = 0.5; ns |
| Conditioned gaping in TR test (f) | 7.8 | (±2.9) | 3.5 (±0.9) | t (14) = 3.2; p = 0.007 |

Data presented = mean (±SEM);
f, frequency;
s, seconds

### Experiment 2: Potential of Oleoyl Glycine and Oleoyl Alanine (HU595) to Prevent Acute Naloxone Precipitated MWD at 10 min and 60 min Prior to Naloxone

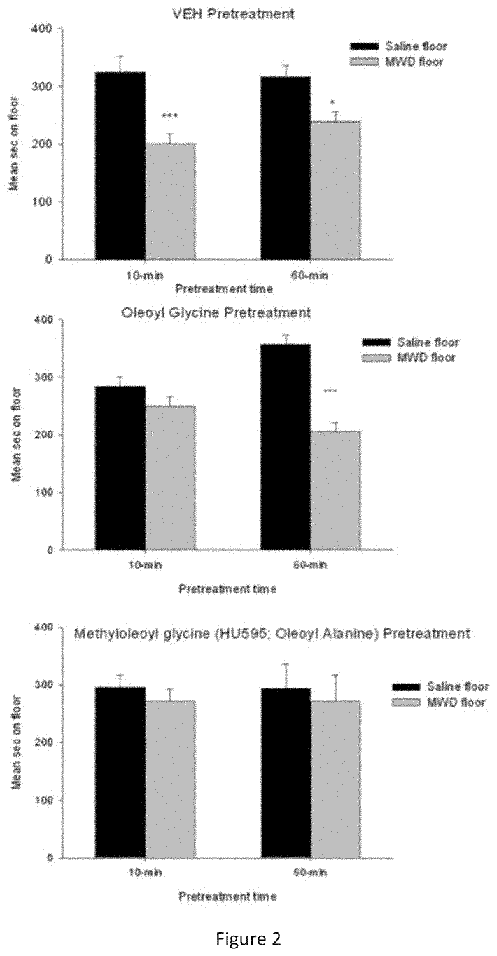

At a dose of 5 mg/kg, i.p., HU595 (oleoyl alanine) produced longer lasting prevention of acute naloxone-precipitated MWD than did oleoyl glycine. FIG. 2 presents the mean seconds spent on the saline-paired floor and on the MWD-paired floor for each of the pretreatment groups. Among the vehicle-pretreated groups (upper section of FIG. 2), the analysis revealed only significant effects of time (F(1,22)=5.3; p<0.031), and floor (F(1,22)=13.9; p<0.001), with the rats at both times demonstrating an aversion to the MWD-paired floor. Among the oleoyl glycine pretreated groups (middle section of FIG. 2), the analysis revealed a significant time effect (F(1,22)=18.1; p<0.001), a floor effect (F(1,22)=17.4; p<0.001), and a time×floor interaction (F(1,22)=7.0; p=0.015); subsequent paired t tests revealed that oleoyl glycine prevented the MWD-induced CPA when given 10 min before naloxone, but not when given 60 min before naloxone. Among the HU595 (oleoyl alanine) pretreated groups (bottom section of FIG. 2), the analysis revealed no significant effects; that is, the rats did not display an aversion to the MWD-paired floor when administered HU595 at either 10 min or 60 min before naloxone. However, when administered 120 min prior to naloxone (data not displayed), oleoyl alanine no longer interfered with the MWD CPA; the rats spent significantly (t (11) 2.6; p=0.026) less time on the MWD-paired floor (M=193.5±29.6 s) than on the saline paired floor (M=355.5±34.1). As was evident with oleoyl glycine (Petrie et al., 2019) at a dose of 1 mg/kg, oleoyl alanine interfered with the MWD (data not displayed); the time spent on the MWD-paired floor (M=264.3±18.3 s) did not significantly differ from the time spent on the saline-paired floor (M=305.2±20.2 s; t (11)=1.1).

### Experiment 3: Potential of Oleoyl Alanine to Produce CPA or CPP

Oleoyl alanine produced neither CPA nor CPP following two pairings with a distinctive floor (data not shown). A paired t test revealed that the rats did not significantly differ in their preference for the oleoyl alanine-paired floor (mean=278±38.0 s) and the VEH-paired floor (mean=281±41.8 s). Oleoyl alanine did not modify the activity level during the conditioning trials; the analysis of the distance traveled on the oleoyl alanine conditioning trials and the vehicle conditioning trials revealed no significant effects.

### Experiment 4: Potential of Dimethyl Oleoyl Glycine (HU596) to Interfere with an Acute Naloxone Precipitated MWD CPA

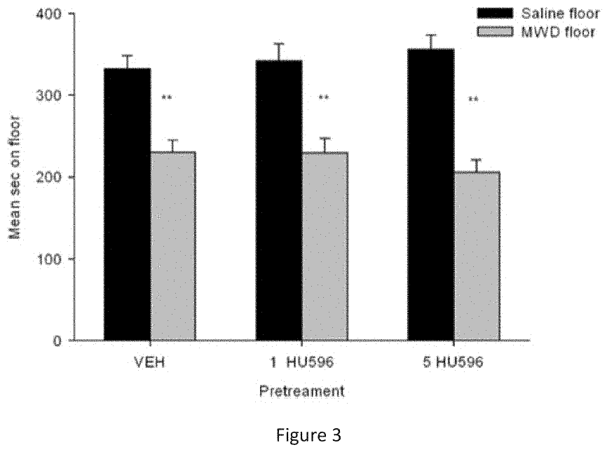

Dimethylated oleoyl glycine (HU596) was ineffective at doses of 1 or 5 mg/kg in preventing MWD CPA. FIG. 3 presents the mean (±SEM) seconds spent on the saline floor and the MWD floor in experiment 4. The analysis revealed only a statistically significant effect of floor (F(1,29)=36.9; p<0.001). All groups displayed MWD-induced CPA.

### Experiment 5: Mechanism of Action of Oleoyl Alanine (HU595) Interference with Acute Naloxone-Induced MWD CPA

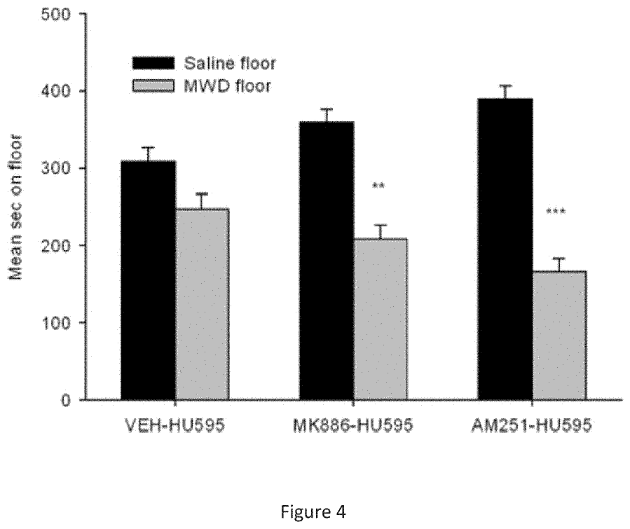

Oleoyl alanine interfered with the MWD CPA, but pretreatment with either MK886 or AM251 prevented this interference as seen in FIG. 4. The analysis revealed a significant effect of floor (F(1,30)=52.2; p<0.001), and a significant interaction of pretreatment group by floor (F(2,30)=4.6; p=0.018). Subsequent paired t tests revealed that groups MK886-HU595 and AM251-HU595 spent less time on the MWD-paired floor than on the saline-paired floor (ps<0.01), but group VEH-HU595 did not show significant CPA.

### Experiment 6: Effect of Oleoyl Alanine (HU595) on Saccharin Preference Following Acute Naloxone Precipitated MWD

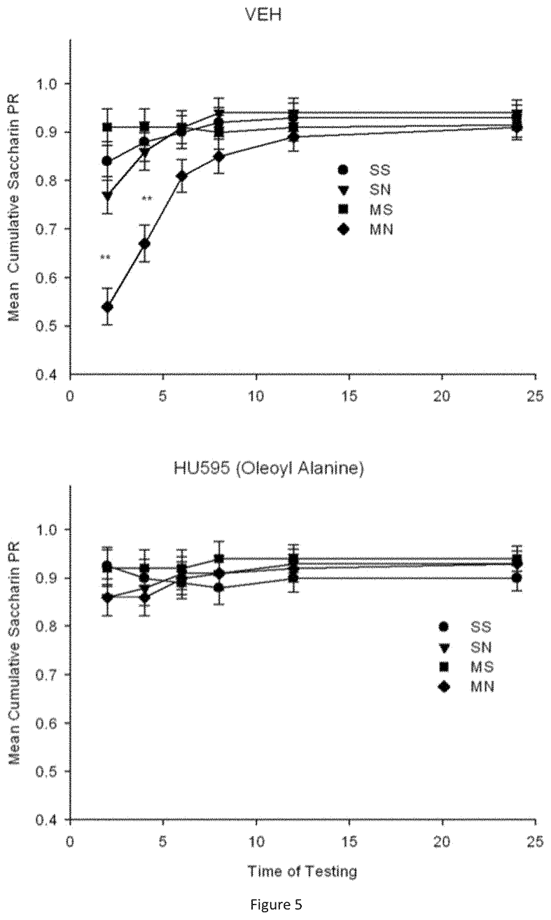

Acute naloxone-precipitated morphine withdrawal suppressed saccharin preference for the first 4 h of the test among VEH pretreated rats, but not among HU595 pretreated rats. FIG. 5 presents the mean cumulative saccharin PR across the 24 h of testing for rats pretreated with VEH or HU595. The 2×4×6 mixed-factors ANOVA revealed significant effects of time (F(5,360)=44.44; p<0.001), pretreatment×time (F(5,360)=18.46; p<0.001), group×time (F(15,360)=11.98; p<0.001), and pretreatment×group×time (F(15,360)=5.07; p<0.001). To evaluate the triple interaction, separate single-factor ANOVAs for the group factor were conducted for each pretreatment condition at each interval. These revealed a significant effect at 2 h (F(3,36)=13.0; p<0.001), 4 h (F(3,36)=8.9; p<0.001), and 6 h (F(3,36)=3.3; p=0.03) among the VEH pretreated groups, but not among the HU595 pretreated groups. Subsequent Bonferroni post hoc comparison tests revealed that among the VEH pretreated groups, group MN displayed significantly suppressed saccharin PRs than all other groups after 2 and 4 h of drinking (ps<0.01).

### Experiment 7: Effect of Oleoyl Alanine (HU595) and HU596 on the Rat Serine Hydrolase Proteome for FAAH and on Luciferase Assay for PPARα

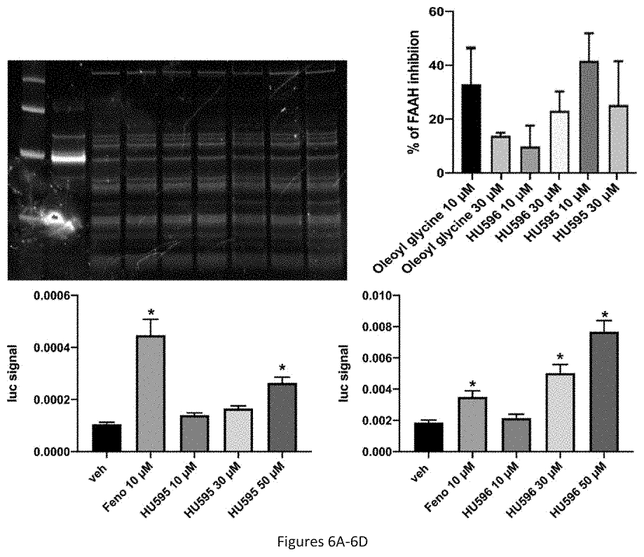

The top section of FIG. 6 a and b shows the effect of two concentrations of oleoyl alanine and HU596 on a rat brain serine hydrolase proteome, including fatty acid amide hydrolase, in comparison with OlGly. Of the three compounds, only oleoyl glycine, in agreement with our previous report (Donvito et al., 2019), and oleoyl alanine inhibited the binding of the serine hydrolase probe to FAAH (by about 40% at 10 without affecting the binding to the other serine hydrolases that can be identified with this method. As shown in FIG. 6 b, the effect of oleoyl alanine at 10 μM was comparable with that of oleoyl glycine, whereas HU596 did not show significant inhibition at either concentration tested. The bottom section of FIGS. 6 c and d shows the effect of three concentrations of oleoyl alanine and HU596 on PPARα activity in a luciferase assay, in comparison with the standard PPARα agonist, fenofibrate. Both compounds revealed PPARα activity at a 50 concentration, but HU596 was more potent and efficacious than oleoyl alanine, with a significant effect being observed at a 30 μM concentration.

Discussion

Oleoyl glycine is a brain lipid capable of reducing nicotine self-administration and morphine withdrawal signs acting via Table 1 Acute naloxone precipitated MWD behaviors in experiment 1 (from Rock et al., 2019). Data presented=mean (±SEM); f, frequency; s, seconds MWD behavior VEH pretreatment HU595 pretreatment Statistical significance Mouthing movements (f) 57.2 (±6.6) 30.8 (±7.4) t (14)=2.6; p=0.025 Abdominal contractions (f) 13.1 (±2.4) 5.0 (±0.8) t (14)=3.2; p=0.006 Lying on belly (s) 722.3 (±120.4) 102.0 (±57.9) t (14)=4.6; p<0.001 Diarrhea (yes/no) 0.5 (±0.2) 0.8 (±0.2) t (14)=1.0; ns % Body weight loss 2.7 (±0.5) 2.4 (±0.4) t (14)=0.5; ns Conditioned gaping in TR test (f) 7.8 (±2.9) 3.5 (±0.9) t (14)=3.2; p=0.007 distinct molecular targets (Donvito et al., 2019; Petrie et al., 2019; Rock et al., 2019). Like other N-acyl-glycines, this endogenous molecule is subject to inactivation by enzymatic hydrolysis (Huang et al., 2001; Donvito et al., 2019). We have investigated here the capability of two methylated, and hence potentially more stable to hydrolysis, analogs of oleoyl glycine on the effects of morphine withdrawal. Oleoyl alanine (HU595) interfered with acute naloxone-precipitated opiate withdrawal when assessed by the somatic and affective effects of MWD (experiments 1, 2, and 6) in male Sprague Dawley. As previously reported with OlGly (Donvito et al., 2019; Petrie et al., 2019), HU595 had no aversive or rewarding effects of its own using place conditioning (experiment 3). As was demonstrated by oleoyl glycine (Rock et al., 2019), oleoyl alanine reduced somatic MWD symptoms and also reduced the display of conditioned gaping produced by MWD. Oleoyl glycine and oleoyl alanine were equally effective in reducing the aversive effects of acute naloxone precipitated MWD at doses of 1 and 5 mg/kg, i.p., when administered 10 min prior to naloxone. However, oleoyl alanine retained its activity for a longer period (60 min, but not 120 min) than oleoyl glycine, in agreement with its potentially higher stability toward FAAH than oleoyl glycine. Hence, we synthesized an additional molecule, the dimethylated oleoyl glycine (HU596), which, we assumed, would cause a larger steric hindrance. However, this chemical change ultimately eliminated activity, possibly for reasons discussed below. Finally, rats experiencing acute naloxone-precipitated MWD also showed the anhedonic reaction of suppressed saccharin PRs, but pretreatment with oleoyl alanine prevented this anhedonic reaction. Interference of acute naloxone-precipitated MWD by oleoyl alanine was reversed by pretreatment with either a PPARα antagonist or by a CB1 receptor antagonist, as previously shown with oleoyl glycine (Rock et al., 2019). These results are consistent with the report that oleoyl glycine acts in vitro as a PPARα agonist and a weak FAAH inhibitor. Petrie et al. (2019), however, reported that the protective effect of oleoyl glycine on acute naloxone-precipitated MWD-induced CPA was reversed by pretreatment with a CB1 antagonist only, suggesting that the mechanism of action of oleoyl glycine on the aversive effects of MWD may be secondary to inhibition of FAAH elevating anandamide and indirectly activating CB1. Here, we found that while oleoyl alanine, like oleoyl glycine, was capable of both inhibiting FAAH and directly activating PPARα at intermediate micromolar concentrations in vitro, HU596 could only exert the latter of these effects at the same concentrations. These findings suggest that, at least in the case of these two compounds, indirect activation of CB1 (via FAAH inhibition and elevation of anandamide levels) is both necessary and sufficient, whereas activation of PPARα is necessary but not sufficient to exert the interference with naloxone-precipitated MWD. This hypothesis would also explain why HU596 is inactive at producing this effect despite its potentially enhanced stability to hydrolysis, whereas it does not contradict our hypothesis that oleoyl alanine has a more prolonged duration of action compared with oleoyl glycine due to its higher stability to hydrolysis. In fact, it is likely that oleoyl alanine still binds effectively to FAAH but, unlike oleoyl glycine, is not hydrolyzed by the enzyme. Alternatively, PPARα may interfere with naloxone precipitated MWD only at a later stage, following FAAH inhibition, which would explain why the less enzymatically stable oleoyl glycine is capable of altering acute MWD in a fashion not mediated by the PPARα receptor. The interference with the aversive effects of MWD by oleoyl alanine by AM251 or MK886 was not the result of an effect of the pretreatment agent itself, as Petrie et al. (2019) showed that at the same doses and under the identical experimental conditions, neither pretreatment drug modified the strength of the acute naloxone-precipitated MWD CPA on their own. Instead, they blocked either the direct (at PPARα) or indirect (FAAH inhibition) effects of oleoyl alanine. The results suggest oleoyl alanine (HU595) may be a more stable agent to combat the aversive effects of acute naloxone-precipitated MWD than oleoyl glycine. At doses of 1 or 5 mg/kg, both compounds prevent the aversive effects of MWD in a CPA paradigm (Petrie et al., 2019), but the preventative effect of oleoyl alanine (HU595) persists over 60 min, unlike that of oleoyl glycine.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A fatty acid amide of an amino acid, including a stereoisomer and a salt thereof for use in the treatment of a patient suffering from nausea including any condition and symptom associated therewith.

2. The fatty acid amide according to claim 1, wherein said fatty acid moiety is selected from a saturated fatty acid moiety, a mono-unsaturated fatty acid moiety and a poly unsaturated fatty acid moiety.

3. The fatty acid amide according to claim 1, wherein said amino acid is selected from glycine, dimethylglycine, alanine, serine, cysteine, tyrosine and phenylalanine.

4. The fatty acid amide according to claim 1, wherein said amino acid is substituted by at least one group selected from straight or branched —C₁-C₆ alkyl, straight or branched —C₂-C₆ alkenyl, straight or branched —C₂-C₆ alkynyl, —OH, and —O(C₁-C₁₀ alkyl).

5. The fatty acid amide according to claim 1, wherein said amino acid is substituted by at least one —C₁-C₆ alkyl.

6. The fatty acid amide according to claim 1, wherein said amino acid is substituted by at least two —C₁-C₆ alkyl.

7. The fatty acid amide according to claim 5 or 6, wherein said —C₁-C₆ alkyl is methyl.

8. The fatty acid amide according to claim 5 or 6, wherein said substitution is on the α-position of said amino acid moiety.

9. The fatty acid amide according to any one of the preceding claims, wherein said fatty acid moiety is substituted by at least one group selected from —C₁-C₆ alkyl, —OH, —O(C₁-C₁₀ alkyl), —SH and —S(C₁-C₁₀ alkyl).

10. The fatty acid amide according to claim 9, wherein said fatty acid moiety is substituted by at least one —C₁-C₆ alkyl.

11. The fatty acid amide according to claim 9, wherein at least one C₁-C₆ alkyl is methyl.

12. The fatty acid amide according to any one of claims 9 to 11, wherein said at least one substitution is on at least one of α- or β-positions of said fatty acid moiety.

13. The fatty acid amide according to claim 1, being a compound of general formula (I), including a stereoisomer and a salt thereof: 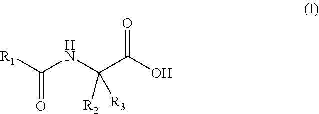
wherein
R₁ is selected from straight or branched —C₁₃-C₂₂ alkyl, straight or branched —C₁₃-C₂₂ alkenyl and straight or branched —C₁₃-C₂₂ alkynyl; optionally substituted by at least one group selected from —C₁-C₆ alkyl, —OH, —O(C₁-C₁₀ alkyl), —SH and —S(C₁-C₁₀ alkyl);
R₂ and R₃ are independently selected from H, straight or branched —C₁-C₆ alkyl, straight or branched —C₂-C₆ alkenyl, straight or branched —C₂-C₆ alkynyl; each optionally substituted by at least one —OH, —SH, —O(C₁-C₆ alkyl), phenyl and phenol;
provided that at least one of R₂ and R₃ is different than H.

14. The fatty acid amide according to claim 13, wherein R₂ is —C₁-C₆ alkyl.

15. The fatty acid amide according to claim 13 or 14, wherein R₃ is —C₁-C₆ alkyl.

16. The fatty acid amide according to claim 13, wherein R₂ and R₃ are each independently —C₁-C₆ alkyl.

17. The fatty acid amide according to any one of claims 13 to 16, wherein said —C₁-C₆ alkyl is methyl.

18. The fatty acid amide according to any one of claims 13 to 17, wherein R₁ is a —C₁₃-C₂₂ alkenyl.

19. The fatty acid amide according to claim 18, wherein said —C₁₃-C₂₂ alkenyl comprises between 1 to 6 double bonds.

20. A method of treating nausea including any condition and symptom associated therewith in a patient suffering therefrom, said method comprising administering to said patient a fatty acid amide of an amino acid, including a stereoisomer and a salt thereof.
